(19) United States
(12) Patent Application Publication
(10) Pub. No.: US 2010/0113152 A1
(43) Pub. Date: May 6, 2010

(54) COMPUTER GAMES BASED ON MENTAL IMAGERY

(76) Inventor: Ron Shmuel, Rishon Lezion (IL)

Correspondence Address: Dr Ron Samuel, 5 Habrosh Street, Rishon Lezion, NA, 75534, omitted

(21) Appl. No.: 12/687,897

(22) Filed: Jan. 15, 2010

Related U.S. Application Data
(63) Continuation-in-part of application No. 12/523,750, filed on Jul. 20, 2009.
(60) Provisional application No. 60/887,159, filed on Jan. 30, 2007.

(51) Int. Cl. A63F 9/24; A61B 5/00

(52) U.S. Cl. 463/36; 600/301

(57) ABSTRACT

A method for playing computer games on a computerized game system having a display, memory and one or more input devices. The method includes one or more acts of pre-game activity and one or more acts of game activity. The pre-game activity includes learning rules of the game including observing one or more objects related to the game on the display, thereby forming a visual percept of the objects, and transforming the visual percept of the objects into a mental image percept of the objects, in the absence of a visual percept of the objects. At the game activity one or more of objects are invisible. The game activity includes activating one or more of the input devices and inputting data in dependence upon the mental imagery percept of the invisible objects, thereby forming play input data. The game activity further includes receiving feedback that corresponds to the play input data and activating one or more of the input devices to form additional play input data in dependence upon the received feedback.

### CROSS REFERENCE TO RELATED APPLICATIONS

This application is a continuation in part of application Ser. No. 12/523,750, filed on Jul. 20, 2009, now pending, the disclosure which is incorporated by reference for all purposes as if fully set forth herein.

### FIELD OF THE INVENTION

This invention relates to methods for playing computer games and particularly to methods for playing computer games having a pre-game activity session and a game activity session, which follows the pre-game activity session.

### BACKGROUND OF THE INVENTION

The aim of a computer game is to entertain, excite, challenge, train, and enhance image perception and the cognitive capacity of the player. Those tasks are achieved by using memory, mental imagery, visual, auditory (two dimensional (2D) and/or three dimensional (3D)) or somato-sensory percept, together with other modalities such as speech, tactile vibration and hand motor activities.

In this invention, during the playing of the game, visual information relating to the game is removed from the display and the player has to base his/her response on alternative means of interpretation and decision making. The term “Mental imagery” as used herein refers to a sensation-like or perception-like experience that occurs in the absence of the stimuli that would ordinary be present to elicit the experience. Mental imagery is a constructive process, but, in contrast to the true sensation mental imagery is much more flexible. The major purposes of imagery are to retrieve information from memory, extract information from visual, auditory and somato-sensory perception and use them to anticipate and execute future tasks. It has been shown that visual percept mental imagery is a significant factor in the retention of location information. Thus, the execution of an activity related to a particular object of the game, when no visual percept of the object is present, depends on the image percept, recalled memory and the recalled memory in association with the task at hand. When the visual percept of the object is prevented, the link between memory and mental imagery makes it possible to supplement the available information. Furthermore, by tying memory and mental imagery, the task at hand can be more easily performed. The integrity and our ability to generate mental imagery are of paramount importance in our daily life. Auditory imagery has mnemonic value similar to that of visual imagery.

The use of mental imagery in games extends the variables used to plan and construct our activities. Mental imagery provides the means for execution of a task with a dimension not available without imagination. For example, we can scale 1D, 2D or 3D dimensions of an object in an imaginary image, or rotate the image percept to our desire. It should be noted that when no vision is present and mental imagery replaces visual percept, the player may inherently introduce an element of flexibility in the sensory percept. To put it differently, if the player has seen the location of an item on the screen of a computer in a certain place, when vision is later absent, mental image is crucial to identify the item and locate it “about” at the imagery perceived location. Usually what is stored in the image does not correspond exactly to the visually perceive physical dimensions. Additional information is needed to exactly locate the item position, or to scale the dimensions of the item. In this invention, the additional information is provided to the player by physiological variables, speech analysis and 1D or 2D auditory signals.

The characterization of resources and limitations in information processing is one of the fundamental problems in cognition. Limitation in task that demands cognition might be apparent when two tasks have to be performed simultaneously. Current theories of dual-task performance have modeled limitations in terms of resources or capacities, outcome and competition conflicts. Limitations emerge when demands must compete for processing capacities. Outcome conflicts might rise where limitations emerge due to confusions or “cross-talk” of parallel processing in response to task demand. Although the task demands might be in competition with one another, it has been suggested that the task demands may, in fact, not be mutually exclusive.

The dual-task performance limitation is apparent in children and adults faced of performing two tasks at the same time. For example, when the first task is motor and the second task requires cognitive efforts. The cost of dual-task performance is also considered to result from the need of “divided attention”. In a non-limiting example, adults and children who have a learning disability, perform poorly on dual-task. Learning at school requires often that the child will divide his/her attention to cope with the teacher and his/her surrounding class. The outcomes in those situations are that children who have a learning disability cannot learn to pay attention to the teacher and the learned material at the same time.

Attention can be divided in several classes, each in need of specific learning and training practice. “Sustained attention” is considered to be a limited factor in adults and children that are hyperactive and cannot concentrate on one task for a long duration.

In an embodiment of the present invention, during the game, the player has to select among several options. In a non-limiting example, the options can be to determine the game rules, or choose a weapon to shoot the opponent with, a choice that might change the game rules. Changing attention options can be done every few seconds when the player has to play for several minutes. The changing of attention options requires player's ability to cope with “alternating attention”. If the player has difficulty to alternate among the options he will perform poorly in such a game. Choosing from a set of options is referred to as “selective attention”.

In the embodiment of the present invention, a set of rules can be set to match the need for training each of the attentions to be addressed. In a non-restricting example, in a game where the player concentrates on the game but in addition, auditory signals are given introducing new rules or modifying the existing rules. It should be noted that “divided attention” is necessary to do the task.

Bilateral motor synchronization is practiced in limbs rhythmic tasks. If the limbs move at the same frequency, it represents “frequency locking”; whereas if they are also phase coordinated (limb move at the same time or at a fixed difference between them) it represents “phase locking”. The coordination phase locking can be either “in phase” or “anti-phase” (limb alternate). Synchronization is greatly affected whether the player has to attend to another task at the same time, for example, to do a cognitive task, and requires divided attention between the two tasks.

When the synchronization is done with the hands, the hands can press the keyboard keys in a repetitive mode either locked and phase locked or anti-phase locked. As in computer games played according to the present invention there is no visual information related to the game, a player response is typically performed by his/her hands, usually on the keyboard using his/her mental image percept. Such games can be both fun and useful training as it is well known that hands motor synchronization training helps also in limbs synchronization, body equilibrium and fine motor activities.

Imagery identifies a specific level of information processing within the percept level at which mental images are functionally equivalent. Mental images can be functionally equivalent to physical objects and events at many levels of percept and can represent percepts and sensory percepts that are not available otherwise. Emotion is probably a good example were mental imagery can estimate the mental state. Emotion gives us some knowledge of the feeling but it is very difficult to place emotion on a continuum scale of mental state. In this patent, when a sensory percept or other feelings are imagined, an additional relative dimension of an auditory signal or physiological levels is given that helps scale mental imagery.

Emotional feeling is considered basically a perception of bodily changes and reflected through measurements in the automatic changes. When measured above some personal level (“baseline”) of emotional state, some of the measurements are considered to indicate generalized stress or arousal rather than particular emotions. Training subjects in relaxation techniques by measuring stress-related variables such as pulse rate, breathing pattern, blood pressure, and skin electrical resistance (galvanic skin resistance) of the palm of a hand or its fingers, is well know. The response of an individual to a verbal provocation which touches him personally is usually related to an emotional reaction with some degree of stress.

The understanding of human vocal emotion has been widely studied in the art. See for example, Murray I. R., Arnott J. L., Toward the Simulation of Emotion in Synthetic Speech: A review of the literature on human vocal emotion, Journal of Acoustical Society of America, 93, pp: 1097-1108, 1993, which is incorporated by reference for all purposes as if fully set forth herein. Murray et al. reviewed the state of the art in our understanding of human vocal emotion. The acoustic properties appearing to be among the most sensitive indicators of emotion were attributes that specified the contour of F0 throughout as utterance, Murray et al. refer to a multi-variable model in which different speech characteristics are associated with emotions such as anger, happiness, sadness, fear and disgust. For example, a faster speech rate is characterized by higher pitch average, wider pitch, higher intensity, abrupt pitch changes and tense articulation.

U.S. Patent application 20040249634, by Yoav Degani et al., describes an apparatus for determining emotional arousal by speech analysis of acoustical and prosodic feature of speech, using speech samples processed into silent and active speech segments including pitch and amplitude parameters. U.S. Pat. No. 5,647,834, given to Samuel Ron and which is incorporated by reference for all purposes as if fully set forth herein, describes a speech based biofeedback regulation system. The biofeedback regulation system enables a subject to monitor and to alter this emotional state based on visual percept of physiological measured variables. An emotional indication is extracted from the subject's speech and compared online with physiological measurements of the subject that serves as a reference for his/her emotional condition. Using visual presentation, the subject can then try to alter the indication signal in order to gain control over emotional state.

Perceptual imagery has also been widely studied in the art. See for example, Mechsner F, Knoblich G, Printz W. Perceptual Basis of Bimanual Coordination, Nature, 414, pp: 69-73, 2001, which is incorporated by reference for all purposes as if fully set forth herein. Mechsner et al. have revealed that perception and perceptual imagery are important contribution to motor coordination in general and in synchronic coordination between the two hands in particular. Bimanual finger tapping was used to show that repetitive equally spaced tones generated synchronized hands tapping that are probably organized in the domain of perception imagery and that voluntary movements are, in general, organized by way of representation of perceptual goal. This would indicate that perception training in general and in perceptual mental imagery would improve both motor movement coordination and synchronization.

U.S. Patent application 20070166675, by Sharona M. Atkins et al., presents an invention for the purpose of enhancing cognition. The participants are required to respond to the presented visual percept. Training is done only in the presence of the visual stimuli that serve as the guide to carry the set of exercises suggested.

None of the above prior art references provide methods that use the mental imagery percept, physiological variables and speech as indicator to replace visual perception in games and training, without the visual perception of one or more objects related to the game activities, when the one or more objects are absent from the display.

It should be noted that there is a large range of “audio games” that consist of sound and have only auditory (no visual output). Audio games are not specifically “games for the blind”. But since one does not need vision to be able to play audio games, most audio games are developed by and for the blind community. Furthermore, most of the audio games do not use their mouse to control their computer. This is because using the cursor to click things instead of tabbing through the different options to reach their goal, is nearly impossible for the blind users, and an unnecessarily challenge for the partly visually impaired.

A principle difference between prior art audio games and the invention is that there is a pre-game training of the player using a visual exposure period, thus guiding the player's responses during the game, when no visual percept of the object (or game) is present, to specific channels that involve visual mental imagery visual percept. The player in audio game has to base his/her answer on mental image auditory percept and relate that to the game. Thus, most of the audio games are very simple games (compared to the mainstream games) and lack the properties of mainstream games such as diversity, multiplayer functionality and good replay ability.

### DEFINITIONS

The term “flash”, as used herein, refers to a time interval that is shorter than the time required between two consecutive eye movements and is long enough for a player to perceive the location of an object on a display, the object being moving or stationary. Typically, the time interval is measured in seconds, and can be adjusted in dependence upon detected physiological parameters of the player. Typically, the time interval is measured in milliseconds, and can be adjusted in dependence upon detected physiological parameters of the player. The flash should be short enough such that the short visual percept enhances the “mental imagery percept”.

The term “eye movement”, as used herein, refers to the time required for an eye motion from one fixation target to a new fixation target, typically hundreds of milliseconds.

The term “flash information”, as used herein, refers to the visual information conveyed to a player during a flash.

The terms “remove” and “vanish”, as they relate to graphical items/objects displayed on the screen, are used herein interchangeably. In particular, the terms “remove” and “vanish” are used herein, refer to the transforming of selected objects from being visible to the player during the pre-game session, to become invisible to the player during the game session.

The term “invisible object”, as used herein, refers to a selected object, relate to the game, that vanish from the screen during the game session.

The terms “screen” and “display”, as they relate to a computerized system on which the game is played, are used herein interchangeably.

The term “prediction”, as it relates to imagery capacity, is used in our daily activities (for example, looking for the upcoming traffic before crossings the road, driving while taking in consideration the traffic or catching a ball). The predictive modality is the ability to predict the outcome of the same or different modality. To help establish the information upon which the prediction is based, one or more flashes can be given to the player. To predict, one has to “anticipate” the outcome or the desired modality. Prediction is always based on memory, psycho-physiological state, visual or auditory percept, sensory modalities or motor activity.

### SUMMARY OF THE INVENTION

A principle intention of the present invention includes providing methods for playing computer games having a pre-game activity session and a game activity session. During the pre-game activity the player learns the rules of the game, including observing one or more objects related to the game on the display, thereby forming a visual percept of the objects, and transforming the visual percept of the objects into a mental image percept of the objects, in the absence of a visual percept of the objects. During the game activity one or more of objects, related to the game, become invisible. The game activity based on mental imagery visual percept includes activating standard computer periphery input device and inputting data in dependence upon the mental imagery percept of the invisible objects, thereby forming play input data. The game activity further includes receiving feedback that corresponds to the play input data and activating one or more of the input devices to form additional play input data in dependence upon the received feedback.

In the absence of visual percept, mental imagery visual percept and memory are the main source for reconstructing the image and improve cognition, motor coordination and predicting ability, attention and concentration, and train the player to handle simultaneously two tasks. The mental image is constructive as it is further used by the player after the game in everyday life activity.

According to the teachings of the present invention there is provided a method for playing games, performed on a computerized system such as a personal computer, a PDA computing device, a portable computing device or a mobile phone. The computerized system includes a display, memory and one or more input devices. The input devices are selected from the group of peripheral devices including a keyboard, a joystick, a mouse, a game pad, a push button and other commonly known computer games input devices.

The method for playing a computer game by one or more players includes one or more acts of pre-game activity and one or more acts of game activity. The pre-game activity includes learning rules of the game including observing one or more objects related to the game on the display, thereby forming a visual percept of the objects, and transforming the visual percept of the objects into a mental image percept of the objects, in the absence of a visual percept of the objects. The one or more objects are selected from the group consisting of stationary objects and moving objects

At the game activity one or more of objects related to the game become invisible for a duration of longer than T_{f}, wherein T_{f} is shorter than a time required for an eye movement between two fixation targets and long enough such that the location of said one or more objects can be visually perceived. The game activity includes activating one or more of the input devices and inputting data in dependence upon the mental imagery percept of the invisible objects, thereby forming play input data. The game activity further includes receiving feedback that corresponds to the play input data and activating one or more of the input devices to form additional play input data in dependence upon the received feedback. The feedback can be provided to the player as an audio signal, a textual message displayed on the screen, turning on/off one or more light indicators—preferably a colored light, a vibration feedback or any other feedback the like.

Preferably, the one or more acts of game activity further includes a flash displayed for a duration T_{f}, wherein T_{f} is shorter than a time required for eye movement between two fixation targets and long enough such that the location of the one or more objects can be visually perceived.

Optionally, the one or more input devices include a microphone, and the act of pre-game activity further includes a step of recording the voice of the player. The voice is then conveyed by the microphone to an analyzer and the memory, thereby creating an emotional state baseline of the player. The voice parameters of the player are selected from the group including: pitch, amplitude, pace, rhythm and prosodic. The emotional state of the player, recorded during playing of the game, is compared with the emotional state baseline of the player, thereby determining the change in emotional state of the player. The game rules can then be modified, using the change in emotional state of the player.

Optionally, one or more physiological parameters related to one or more players' physiology, are monitored in real-time and wherein the game rules are modified in dependence upon the physiological parameters.

In variations of the present invention, the acts of game activity further include the step of selecting and operating at least one of the input devices by the player, in particular in games that demand performance of dual-task.

In variations of the present invention, the acts of game activity further includes the step of measuring at least one physiological monitoring tool selected from the group including: monitors of respiratory rhythms, heart rate, blood pressure, tactile, resistance skin conductance, encephalogram and electro-myograph.

Optionally, the acts of game activity further includes the step of providing each of the players with a flash of visual percept of the display, and wherein the flash is activated by the player, randomly by the game or at the occurrence of a predetermined event in the game.

In variations of the present invention, one of the players is the host computer.

In embodiments of the present invention, an objective of the game is to hit a target, for example with a missile, wherein the target can be a stationary or a moving target. Speakers are activated to sound auditory signals associated with the display spatial location of the target. The player operates at least one of the input devices to activate a weapon to launch ammunition to hit the target, for example with a missile. The target is selected from the group consisting: ground targets, air targets, outer-space targets and underwater targets.

In embodiments of the present invention, the computer game is a puzzle game having a character associated with the player. The puzzle is rotated (with respect to the orientation in the pre-game activity) and optionally changes position on the screen (with respect to the position in the pre-game activity) and optional scaled (with respect to the dimensions in the pre-game activity), before the acts of game activity begin. The acts of game activity further includes the steps of: providing the player with different pre-stored questions and phrases such as syllables, words, items, pictures, auditory grapheme or phoneme and sounds to increase the skills in learning in general and learning languages in particular; and providing an answer by the player, wherein the correct answer is provided to the player by an auditory signal immediately after the answer of the player or at the end of the game.

In embodiments of the present invention, the computer game is a drawing game having a drawing pointer associated with the player, wherein the acts of game activity further includes the step of operating at least one of the input devices by the player to activate the drawing pointer to draw a drawing task without visual percept of at least a portion of the drawing. The drawing task includes changes in the drawing, selected from the group including dimensions and orientations.

In embodiments of the present invention, the computer game is a racing game between two or more of the players, and wherein said one or more object includes a racing object associated with each of the players, wherein the acts of game activity further includes the step of operating at least one of the input devices by each of the players to activate the racing objects. The racing object is selected from the group vehicles and animals. The vehicles are selected from the group including land vehicles, air vehicles, outer-space vehicles and water vehicles. The acts of the racing game activity may further includes the step of activating speakers to sound auditory signals, wherein the auditory signals are respectively associated with the spatial location of the racing objects, and wherein the auditory signals are facilitated to change in intensity, pitch, or frequency. The acts of the racing game activity may further include the step of activating a vibrator to provide tactile information, the tactile information associated with the spatial location of the racing objects.

In embodiments of the present invention, the computer game is a ball game having a character associated with the player, wherein the acts of game activity further includes the step of operating at least one of the input devices, by the player, to activate the character to bounces the ball with a controllable force to reach a target location, and wherein the surface on which the ball is bouncing has different and changeable elasticity properties.

In embodiments of the present invention, the computer game is a maze having obstacles and a character associated with the player, wherein the maze is rotated (with respect to the orientation in the pre-game activity) and optionally changes position on the screen (with respect to the position in the pre-game activity) and optional scaled (with respect to the dimensions in the pre-game activity), before the acts of game activity begin, and wherein the acts of game activity, further includes the step of operating at least one of the input devices by the player, and wherein the obstacles force the character to walk, bent, run, jump, crawl, swim or shoot in a certain order and certain direction.

In embodiments of the present invention, the computer game is a combat game having a character associated with the player. An objective of the game is to hit the character associated with the opponent player. Speakers are activated to sound auditory signals associated with the display spatial location of the characters. Each player operates at least one of the input devices to select a weapon, to be carried by a respective character of the player. Each player operates at least one of the input devices to activate the selected weapon. Each player operates at least one of the input devices to activate the character to move, including moving, jumping, crawling, swimming, air parachuting or hiding; and wherein said combat situated in a space domain selected from the group consisting: on the ground, at the air and underwater.

An aspect of the present invention is to provide a method for providing a computer game to one or more players, performed on a computerized system such as a personal computer, a PDA computing device, a portable computing device or a mobile phone. The computerized system includes a display, memory and one or more input devices. The input devices are selected from the group of peripheral devices including a keyboard, a joystick, a mouse, a game pad, a push button and other commonly known computer games input devices.

The method for providing a computer game to one or more players includes the step of presenting one or more of the players with pre-game activity information and presenting one or more of the players with game activity. The pre-game activity information includes information related to rules of play, including displaying selected one or more objects on the display for a duration sufficient so that the player can acquire a mental imagery precept.

During game activity, the display does not include information related to the position or movement of the selected objects for a duration of longer than T_{f}, wherein T_{f} is shorter than a time required for an eye movement between two fixation targets and long enough such that the location of said one or more objects can be visually perceived. The game play activity includes the steps of receiving from the one or more input devices input data related the mental imagery percept, providing the player with feedback that corresponds to the input data, receiving additional input data after providing the feedback, and providing the player with additional feedback that corresponds to the additional input data.

Optionally, the game play activity further includes the steps of providing flash information related to the position of the selected object. The feedback can be an audio signal, a textual message displayed on the screen, a light feedback—preferably a colored light, a vibration feedback or any other feedback the like.

During the pre-game activity the player learns the rules of the game, including observing one or more objects related to the game on the display, thereby forming a visual percept of the objects, and in the absence of visual percept of the objects, transforming the visual percept of the objects into a mental image percept of the object.

During the game activity one or more of objects are devoid. The game activity based on mental imagery visual percept includes activating standard computer periphery input device, thereby inputting data in dependence upon the mental imagery percept of the devoid objects, thereby forming play input data. The game activity further includes receiving feedback that corresponds to the play input data and activating one or more of the input devices to form additional play input data in dependence upon the received feedback.

In the absence of visual percept, mental imagery visual percept and memory are the main source for reconstructing the image and improve cognition, motor coordination and predicting ability, attention and concentration, and train the player to handle simultaneously two tasks. The mental image is constructive as it is further used by the player after the game in everyday life activity.

### BRIEF DESCRIPTION OF THE DRAWINGS

In order to understand the innovation and to see how the same way may be carried in practice, some preferred embodiment will now be described, by way of non-limiting examples only with reference to the accompanying topics and examples, in which:

FIG. 1 is a schematic illustration of a system for playing computer games according to the present invention, the system including a computer and accessories;

FIG. 2 shows a bouncing game which is practiced during the pre-game activity and then played from mental imagery visual percept during the game, according to embodiments of the present invention;

FIG. 3 shows a basketball game which is practiced during the pre-game activity and then played from mental imagery visual percept during the game, according to embodiments of the present invention;

FIG. 4 shows an example 3×3 magic square which is revealed and memorized during the pre-game activity and then reconstructed during the game, according to embodiments of the present invention;

FIG. 5 shows a roulette of numbers which are revealed and memorized during the pre-game activity and then reconstructed during the game, according to embodiments of the present invention;

FIG. 6 shows a terrain walking to a target game which is practiced during the pre-game activity and then played from mental imagery visual percept during the game, according to embodiments of the present invention;

FIG. 7 shows a maze game which is practiced during the pre-game activity and then played from mental imagery visual percept during the game, according to embodiments of the present invention;

FIG. 8 shows a track moving game which is practiced during the pre-game activity and then played from mental imagery visual percept and memory during the game, according to embodiments of the present invention;

FIG. 9 illustrates a redrawing game showing a few example images which are revealed during the pre-game activity and then redrawn from mental imagery visual percept and memory during the game, according to embodiments of the present invention;

FIG. 10 illustrates a game with missiles that are trying to hit moving targets, practiced during the pre-game activity and then replayed from mental imagery visual percept during the game, according to embodiments of the present invention, whereas the moving target position is indicated by auditory cues; and

FIG. 11 illustrates a game according to embodiments of the present invention, in which the player has to convert two dimensional auditory signals to a mental imagery visual percept.

### DETAILED DESCRIPTION OF THE INVENTION

The present invention now will be described more fully hereinafter with reference to the accompanying drawings, in which preferred embodiments of the invention are shown. This invention may, however, be embodied in many different forms and should not be construed as limited to the embodiments set forth herein; rather, these embodiments are provided, so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The methods and examples provided herein are illustrative only and not intended to be limiting.

By way of introduction, the principle intention of the present invention includes providing methods and system to play, train and improve players' brain ability to educational, cognitive, and neurological functions by substituting visual percept with mental imagery of auditory and visual percept and other sensory information to be used as feedback during the game.

A principle aspect of the present invention is that before the game starts, visual percept is present for a predetermined time interval and the player learns the rules and options that the game provides. During the game visual information relating to the game is removed, thus there is an absence of the visual game information. That is, one or more objects related to the game and affecting the player's decisions and activities relating to the next act of game activity are removed from the screen. The player's actions are based on other modalities such as feedback to the player, corresponding to an action previously taken. The feedback can be given to the player by means of mental imagery visual auditory or somatic. Playing the game might require all or part of the modalities of auditory and mental imagery visual percept, memory, hand movements, speech, physiological measurements and prediction of hand motor coordination. Optionally, during the game, the player is provided with occasional flashing of game related information, including information previously removed.

It should be noted that when visual information relating to the game is removed, also one or more objects that do not affect the player's decisions and activities relation to the next act of game activity, might also be removed from the screen. It should be further noted that when visual information relating to the game is removed, the screen may by darkened or colored by one or more colors or presented with information not relevant to the game played.

In the pre-game activity, different stimuli are presented so that the player can learn most of the tasks required to answer the game challenge. The system also teaches the player how to respond and adapt the game to his/her capacity. The adaptation can be controlled by the player and increase or decrease his/her challenge and expected scores. Optionally, during the pre-game activity, when vision is present, the player may response by using one or both hands. Then, during the game, the player is aware of his/her previously hand movements (motor template). If no hand movement is present during the pre-game activity, hand movements accuracy during the game depends on the mental imagery percept and is expected to be lower.

The rules of a game are presented to the player during the pre-game activity using auditory signals, visual presentation, physiological and speech variables. Game difficulty level can be determined either by the player or automatically based on player's response level. Auditory signals can be speech or tones that are determined in the game rules. The game can be based on one fixed visual stimulus and a single tone, or a sequence of stimuli and short tones presented sequentially or continuously that evokes the auditory and visual mental image percept. The game can be “dynamic”: during the pre-game activity, a visual stimulus can be one or more moving objects and auditory signals, discrete or continuous, and during the game the audio response is continuous or discrete. The game can be “static”: during the pre-game activity, the visual stimulus can be one or more stationary objects and auditory signals, discrete or continuous, and during the game the audio response is continuous or discrete. The player can train and practice a game of the present invention during the pre-game activity.

Optionally, before the game or at the beginning of the game, the player is asked to speak for a short period, thus providing the computer software with one or more speech samples to be analyzed and recorded, as an additional variable. The speech samples can then be used during the game to track changes in the player's emotional state or the player's arousal. The player is using response variables means such as push bottoms, mouse, joystick, keyboard buttons or touch screen, depending on the particular game version.

During the game, visual information relating to the game is removed, thereby forcing the player to use audio stimuli, mental imagery percept, memory, sensory modalities and tactile vibration, hand motor activities and converting values between psychological, physiological, physical and mathematical modalities. The next player's action can be based on feedback provided to the player, in response to an action previously taken. During the game, the auditory signals can be instructions learned during the pre-game activity or new rules that modify the game configuration. The auditory signals can also be non-related to the game and of content that affect or modify the player's arousal and emotional state.

Auditory signals can be short or continuous and can be of different intensities, pitch and characteristics, speech, animal sound or musical instruments. The changes in the auditory signals can introduce new options to modify the player response requirement. In a non-restricting example, to comply with the game rules, increasing tone pitch might require the player to respond faster or slower for lower pitch. Reaction time can be obtained from moving the mouse, the joystick or pressing the buttons on the keyboard.

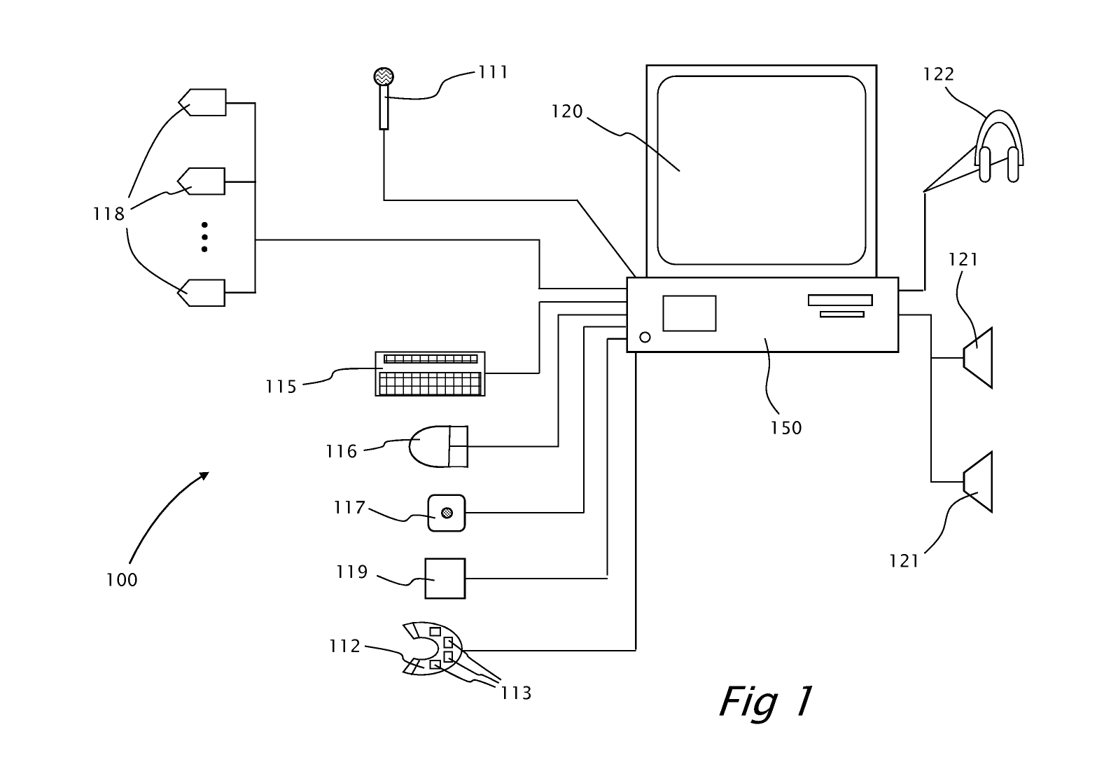

The computer games of the present invention are played on a computerized system. Reference is made to FIG. 1, which is a schematic illustration of system 100 for playing computer games according to the present invention, whereas system 100 includes computer 150 and accessories. Computer 150 can be any standard computer such as a personal computer (PC), or a palm computer, a laptop computer, a personal digital assistance (PDA), a portable computing device, or any other computerized platform. Computerized system 150 includes a display unit 120, used mostly at the pre-game activity, one or more loudspeakers 121 and/or headphones 122 and standard peripherals such as keyboard 115, mouse 116, joystick 117 (with or without force feedback option), a tactile stimulator (vibrator) 119 a microphone 111 for picking up speech signals, and a set of push buttons 113 for example on a game pad 112. Loudspeakers 121 are used for generating sounds during the pre-game activity and the game itself, sounds that are pre-recorded verbal instructions, game rules and clues, or signals that represent the analyzed speech and physiological analyzed data, which can be used as feedback during the game. Loudspeakers 121 should be placed at a balanced distance from the center of screen 120, thereby generate a two dimensional sound (stereo mode), and preferably at a larger distance from screen 120 and directed towards the player.

The game can be played on any standard computer that has standard accessories. However, if more than one player is involved, the game can be played with a game server facilitating the transfer of the program and data to any computer or storage medium via public networks, data dedicated circuits or satellite circuits. For two or more players, the peripherals are duplicated. The two players do not have to be physically close and they can play in different locations such as over remote communication, line phone, wireless devices or Internet. In general, when the game is played over a network, during the game, selected objects are removed from screens 120 of all players. It is possible to play an interactive game where one of the players is a tutor, that during the game, receives visual display of the selected objects, but the student does not. This will allow the tutor to concentrate and guide the player more efficiently.

In embodiments of the present a game is played on a mobile phone and/or a line phone, connected to a remote server or the Internet and speaker or the headphone, and whereas the microphone of the phone is used during the game for detecting speech. Optionally, the phone buttons are used to play the game.

Computer 150 is configured to receive speech characteristics from the digitized speech signal and may include a speech analyzer, such as a DSP (Digital Signal Processor), for speeding up the process of detecting those characteristics which are sensitive to, and indicative of, changes in the emotional state of the player. Computer 150 may further include a plurality of different sensors 118 for detecting parameters such as heart rate, blood pressure, galvanic skin resistance, and so on.

Evaluation of speech and physiological variables are considered to be correlated with arousal and emotional state. If detected during the game, the changes in the player's emotional state from the measured values before the game (“baseline” level) can be relayed to the player via auditory signals, or can be used to maintain or change the game playing conditions. For example, elevated heart rate is considered to indicate stress, high arousal. When this is coupled with low galvanic skin resistance it indicates anger. Thus, depending on the game goals and expectations, the player can maintain his/her emotional state baseline as measured before the game, or for example, the data information can modify the game rules to lower game demands and thus lower the player's tension.

Attention is a complex modality influenced by many factors such as concentration, ability to suppress visual or auditory percepts or hand movements. When attention is required during the presence of two tasks and the player is not able to perform them simultaneously, the player is often handicapped and the performance of the player is low.

When two tasks are presented to the player, for example, a cue demanding a hand motor task and an auditory signal for performing the second task, special attention is needed. The auditory signal can be a signal or speech that requires cognitive decision, special attention for information such as changing the game rules or a story that the player needs to proceed with the game. The player responses might be generated serially, one task after the other that might not satisfy the game demands, or simultaneously, as the player is expected. In the latter, divided attention is required to complete the two tasks. In a non-restricting different example, to answer to two different tones simultaneously in a repetitive fashion; each tone determines one hand response.

In embodiments of the present invention, the player received two signals that require different responses. The responses can be both logical, thus presenting the player with a guessing task, or be of different nature when only one is logical while the other is not. The player has to choose which one is more logical thus performing a selective attention. If the player has to make choices every few seconds, each time concentrating on different items, the player might have to perform an alternate attention. For continuing to perform a dual-task, sustained attention is requires

The lack of ability to perform dual task greatly affects the capabilities in some impairments such as dyslexic patients and subjects that suffer from learning disabilities. They are not able to cope with simultaneous tasks such as concentrating on what the teacher explains and writes on the board and at the same time inhibit (or answer to) the nearby child that talks and destruct his/her attention.

Among modalities which one has to predict in order to complete a task is to evaluate the time on which prediction is based, either single or continuous time events. Following are two classes of prediction based characteristics, with no limitation on the listed classes. The classes are:
a) single event (for example, crossing the road, catching a ball); and
b) repetitive, equally spaced in time events (for example, synchronize a movement such as tapping to coincide with the sound of a repetitive target).

In both predictions based characteristics, the player has to predict the time at which the event will take place and generate a neural signal so that the execution of the predictive response will coincide with the desired event.

Emotional state can have an immediate impact on speech characteristic and to a lesser or slower degree on other physiological variables. In embodiments of the present invention, speech analysis evaluation is incorporated as an extended dimension to evaluate players' emotional state during the game.

Subject emotional reaction can be modified, among other factors, by visual or auditory percept and mental imagery. The player emotional state can be evaluated before or at the beginning of the game, referred as “baseline” emotional state, and during the game it can be maintained or modified depending on the task. The player is taught to maintain or modify his/her expressed speech emotion, based on auditory mental imagery percept state learned during the pre-game activity or beginning of the game, and depending on the auditory instructions.

Memory and mental imagery recall might be based on the information of access to visual and auditory percept. When the percept is present, the information, in principle, can be reproduced. When a partial picture is shown to the player, then, based on memory and previously recalled visual percept during the pre-game activity, the player can complete the picture or draw the whole picture on a blank screen. Auditory cues can be provided depending on the difficulty to form a complete mental imagery.

In embodiments of the present invention, one or more auditory signals are provided to guide the player during the game. During the game activity, to make the player aware of the location of invisible objects on the screen, special sounds might be attached to each object. For example, stationary items might be associated with short repetitive low intensity tone, while moving objects (if they do not have their own sound, for example, a car) can be associated with continuous sounds.

There is a tremendous need for learning languages, vocabulary, spelling in general and foreign language in particular. Furthermore, in their native language in selected groups of student, mastering the grammar is a difficult problem. A crossword puzzle in which the puzzle is not seen, thus the specified word space is not given, poses a challenge and a fun way to learn a language.

In addition to the benefit from addressing a mental imagery visual percept, puzzle solving requires mental reasoning over and above simple memorizing. Thus, the method of playing without visual percept is a challenge by itself, but mental image visual percept improves his chances using a different method to master language learning. If the player misses the correct answer, using the sound signals the player can either be asked to correct the answer, or given the answer in words, vowels or letters and asked to type it on the keyboard. Thus, the game becomes a teaching session of language comprehension.

Synchronization between motor activities of the two hemispheres is not always a straight forward fact. Specific games can be developed to promote such operation involving bilateral operation from the player; such as pushbuttons, keyboard, or two joysticks for one player. The activity has to be coordinated between the two hands or fingers from both hands, such that they will be activated simultaneously or sequentially. The action can be a singular act, or repetitive acts equally spaced in time. The activity can be equally divided between the two hemispheres, or can have different repetitive (yet synchronized) tasks between the two hands. To put it differently, hands coordination in pressing the keyboard keys can be synchronized and move at same frequency at the same time or alternatively, and they can act in-phase or in anti-phase. If the player responses are within an accepted range, then the game can be carried on. If the player misses the frequency or the phase between the two hand, two different tones can be given, one indicating the frequency range and one the phase.

As a non-restrictive example of coordination, in a game which its object is drawing direct lines, curves or shapes or mixture of them, the player has to operate one joystick with each hand. Each of the joysticks is restricted to move in one direction, for example, horizontal or vertical, thus, to move along a diagonal line it requires the coordination of both hands.

In embodiments of the present invention, computer 150 includes a vibration feature. The additional cues provided by the vibration feature are detected by the somato-sensory system that can be incorporated in the game rules. In a specific non-limited example, in the puzzle game where the player has to write a word in syllables, in addition to the auditory signal that tells the player the word to be written with the keyboard, the vibrator might indicate him how to divide the word in syllables. Optionally, the auditory signal delivers the words, in syllables, but it's more effective giving the player the uninterrupted word and additional clues of the syllables. It's advantageous that the additional variable is in principle different that the first signal, (auditory signal and vibration) as the ability to take notice of two different variables is higher than to provide the two signals with one sensor variable. In addition, operating on two different percepts evokes different brain activities. The invention should not be limited to the specifics of these examples.

A given scenario cannot always be associated with the emotional topic to which the scenario is believed to be related, as it is well known that what might be a relaxing scenario for one subject could be a stressful scenario for another. Using the pre-game activity, the player can choose from the presented list auditory scenarios that are associated with a specific emotion such as relaxation, stress, anger sadness, fear, and disgust. The player can imagine a personal event related to the chosen scenario topic.

The auditory sounds can be set independent of the mental imagery perceived emotional topic but, preferably, the prerecorded sounds are adapted to produce sounds considered by most subjects to be related to a specific emotion and to have some emotional effect. For example, a soft playing music can be employed to represent happiness or relaxation, whereas a thunderstorm can represents anger.

In order to allow the fact that some people cannot speak emotionally but his/her emotional state is high, computer 150 may include a plurality of different sensors 118 for detecting parameters such as heart rate, blood pressure, galvanic skin resistance, breathing and so on that might detect higher emotion and convey the output to signal indicating the player how to increase his/her emotion in speech.

Using sensors 118, a player can play the game and maintain the desired emotional state. Different sound characteristics can be related to different emotions. For example, increase pitch can be employed to represent anger, whereas decrease pitch relaxation, increase intensity happiness, whereas lower intensity sadness. Once the player is aware how these emotions can be changed, the player switches to speech and by generating the similar feeling as before, the player learns to change the speech characteristics so that these feelings will reflect during playing the scenario that is emotional related.

In another non-restricting example, while the audio signals can evoke fear, the player can feel fear or anxiety but the speech characteristic might not show the typical moderate increase in pitch associated with anxiety but, rather faster pitch rate associated with fear. The player can control his/her fears, thereby reducing his/her heart rate and thus changing the content, wholly or partially, of the audio scenario to a relax one, thus influencing the playing outcome. Alternatively, the subject can, by changing his/her voice, modify the audio scenario so as to reflect fear, if he so wishes.

The system can also be used wherein the object is for the player to assume a desired emotional state such as being relaxed or excited. To win or “score”, the player has to change his/her speech characteristics so that they reflect desired emotional state. The goal of such a game could be for example, to teach a player to experience a particular emotion and to proceed with the game only when the desired emotion is correctly represented in his/her speech characteristics as a mean to control their emotions and expressions.

Typically, when the game starts, visual information relating to the game is removed and the player imagines a desired emotional state scenario. The player must then relate a personal event corresponding to the displayed image so that the player's speech characteristics truly reflect the nature on the mental imagery. For example, if the scenario is “scary”, the player's voice should express fear, whereas a sad event should be expressed by a sad voice. The “score” is based on the change in the player's speech characteristics. Several different emotional responses as well as skill levels may be associated with the event script so that the player can develop various speech characteristics, each corresponding to a respective emotion. For example, he can increase the intensity, speed and pitch to indicate anger, or monotonic slower voice to indicate relaxation. Once the player has completed a scenario before the game has been completed, to remain in the same framework of emotion, the player can start a new scenario representing the same desired emotion, or change to a scenario which represents a different emotion. The game is both entertaining and enhances the ability of the player to exercise control over the player's emotional expression in speech. Another illustrative example is a racing game in which there are two players playing a competitive game were one player is computer 150. The speed of one racer is determined by computer 150, whilst that of the other is determined by the emotion the subject is asked to express in the voice.

The analyzed speech used in the game can be conveyed to the player through the auditory signal by tones delivered through the loudspeakers. The sound heard by the player can be a tone or short burst whose frequency changes based on the emotional state of the player's voice indicating the player's emotional state. Such pre-recorded evaluations that might be refereed as “baseline” emotional state can be used to modify game rules, instructions dictated to the player from the data storage of computer 150.

The player is instructed that in order to reduce the anger level the player should aim at reducing the rate of the sound burst, or should aim at a lower tone. For example, when the player is angry the rate of the burst is typically one per second (or a tone at 3000 Hz); whereas when the player is relaxed the rate is typically reduced to one burst every three seconds (or 500 Hz). By such means, the player can receive the feedback signal through loudspeaker 121 or headphones 122. It is possible to store the progressive training lessons and the results of the training lessons can be stored in the data bank, so as to allow any improvement in the player's response to be relayed at the end of the training games.

For some players, the speech might not reflect the emotional state as the player may be exited but the player's speech is monotonous with the low pitch indicating a relaxed state. Such players may either not be aware of their emotional state as reflected in the physiological measurements, or they might not know how to speak emotionally. The training of such players are provided by a plurality of sensors that measure such variables as heart rate, blood pressure, breathing, galvanic skin resistance, encephalography or electromyography, known to be influenced by emotional state.

When during the game physiological variables are recorded in addition to speech, the training starts only when the game advances, the subject speaks and one or two physiological variables may be measured simultaneously. If for example the player is under stress as indicated by the galvanic skin resistance but the analysis of the player's speech does not reveal it, the player is told through an audio signal that he does not express the player's emotions. Depending on the task, the subject can be encouraged either to lower the player's stress by giving him the heart rate or galvanic skin resistance measurements as an auditory feedback signal, or be given a feedback signal correlated with the emotional state on the player's speech characteristics and asked by an audio signal to increase the player's emotional reaction. It is apparent that if the aim of the game is to play with the player emotional state, the game is chosen and related accordingly and it serves to enhance the player's reaction.

It should be clear that a principal intention of the present invention is playing a computer game without some predetermined essential visual information related to the game. During the game, the player is required to perform an act of playing the game, and the measured physiological variables and/or speech parameters are used to provide the payer with feedback corresponding to his/her execution of the game related activity. During the game some predetermined essential visual information relating to the game is removed. The actions performed by the player are based on mental imagery and feedback provided corresponding to the action taken. The feedback provided to the player can be auditory, visual clues, vibration of a selected input device or occasional flashes of at least a portion of the game related information previously removed. The visual clues may include textual data displayed on the screen, turning on/off a light indicator, preferably a colored light and other visual clues commonly used.

Optionally, the feedback reflects the monitored psycho-physiological state of the player, such as speech parameters, respiratory rhythms, heart rate, blood pressure, tactile, resistance skin conductance, encephalogram and electro-myograph.

The present invention now will be described through some example games. This invention may, however, be embodied in many different games flowing the same principles, and should not be construed as limited to the example set forth herein; rather, these example are provided, so that this disclosure will fully and clearly convey the scope of the invention to those skilled in the art.

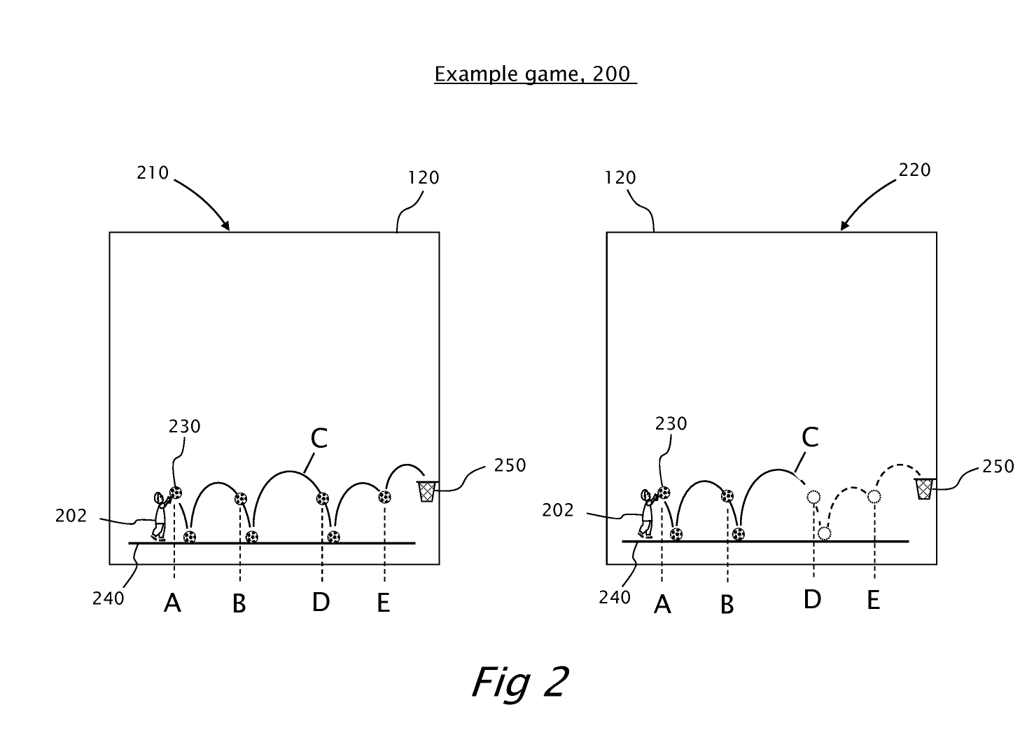

In embodiments of the present invention, the game is a ball game, which can be any ball based game such as basket ball, squash, tennis, etc. Reference is made to FIG. 2, which shows an example of a bouncing ball game 200, according to variations of the present invention. Game 200 is practiced during the pre-game activity, as shown in panel 210, where screen 120 is on and all the game objects are visible. During the game, at some point the image of ball 230 is removed from screen 120 and the player places the ball location on the screen using mental imagery percept, as shown in panel 220. In the example shown in FIG. 2, the player is represented by character 202, whereas the objective of the game is to get ball 230 to pass through ring 250.

During the game, the player has to guess the time to throw ball 230 and thereby reach ring 250, based on the time between the bouncing, the hitting strength on ball 230 (reflects in the time between the bouncing), changing condition of elasticity characteristic and the distance of ball 230 from ring 250. The player has to bounce ball 230 from one end of the court to the other end. Bouncing depends on the strength of the force ball 230 is hit and the elasticity of surface 240 of the ground. Thus, the hitting of ball 230 has to be just right so that in the up direction, ball 230 will reach the height of character 202 hand. If surface 240 is made of materials having low elasticity and ball 230 is hit too strong, ball 230 will bounce too high and too fast and thereby the player will miss ball 230; if ball 230 is hit too weak at surface 240 having the same elasticity, ball 230 will bounce below the hand of character 202 and the player has to lower the hand position to reach ball 230. Thus, the key to easy, fast and accurate play is to control the bouncing of ball 230. When the player starts game 200 the playground elasticity is indicated by a tone; stronger elasticity by a higher pitch and a lower elasticity by a lower pitch. When ball 230 touches surface 240 a tone with different pitch is given indicating to the player that ball 230 has hit surface 240.

The hitting of ball 230 can be performed by pressing a button on keyboard 25 or mouse 26 or game-pad 112 or any other button. The strength at which ball 230 is hit can be set by a number selected on keyboard 115, or by an up or down movement of joystick 117 or any other means.

In the example shown in pre-game panel 210, the player hits ball 230 at point “A”, and preferably, also at point “B”. When ball 230 bounced after point “B” and reached point “C”, game 200 starts, the image of ball 230 vanishes from screen 120 and the player has to hit ball 230 based on the visual perceived imagery at point “D”, and then again in up direction toward ring 250 at point “E”. Every time ball 230 hits playground 240 a feedback tone is heard to indicate that ball 230 has hit surface 240. Obviously, the player should be aware of the tone sound as the player has to hit ball 230 before hitting surface 240, in order to enable ball 230 to make the next bounce. Preferably, the player can control the strength at which ball 230 is hit, which can be set by a number selected on keyboard 115, or by an up or down movement of joystick 117 or any other input means. The time between two tones from consecutive bouncing indicates the height of the bouncing; the longer the time is between bouncing, the higher is the maximal height of ball 230, hence the player can plan the next hit based on the time passed from the last bouncing.

Optionally, the player is helped at the last throwing of ball 230 (point “E”) by providing a special tone. Thus, hearing the special tone of the last bounce before point “E” and the special tone at point “E” provides the player with information to direct the hit strength in the generally up direction, towards ring 250. Optionally, if ball 230 does not enter ring 250, the last throwing is repeated several times and when ball 230 does enter ring 250, a special tone is heard.

In variations of the present invention, when game 200 starts, in addition to ball 230, character 202 also vanishes from screen 120. In other variations of the present invention, when game 200 starts, only character 202 vanishes from screen 120.

In embodiments of the present invention, if the player makes more than a pre-defined number of mistakes, the same game conditions are repeated. If the game ends with a lower than the pre-defined number of mistakes, the player is informed, and new, harder conditions are provided in the next round of game 200. For example, the elasticity of surface 240 will be changed in the middle of game 200, but not indicating in which direction. Thus, the player has to try and find out if the elasticity increases or decreases.

When the player has to play by using both hands in a coordinated fashion either simultaneously, synchronously, or sequentially, the act between the two hands has to be coordinated. In a non-restricting example, a game is played were a line has to be drawn in a diagonal direction. Assuming that each joystick can be moved in one direction only, vertical or horizontal, if the two joysticks have to be moved to obtain a diagonal line, the movement of the left and right hand has to be coordinated and move at the same rate. It should be noted that drawing a diagonal line is given by way of example only, and the scope of the present invention includes drawing tasks that require the player to draw a particular shape, whereas the player can move the drawing pointer only in horizontal direction using one hand, and whereas the player can move the drawing pointer only in vertical direction using the second hand, wherein the shape is selected from the group including: a straight diagonal line, a circle, a polygon or any other 2D shape.

Synchronization between the hands movement is also needed were the game involves player answers tapping on the keyboard buttons in a very regular manner. In another game, the tapping sequence that serves during the game can be made alternatively with each hand at equal spacing, twice with one hand and once with the other or tapping with each hand at different time spaced intervals. Such responses might be required, for example, if in a shooting game the player has to shoot in a repetitive equally spaced manner when the army of the opponent is exposed only for equal short intervals. To hit the opponent solders that are located in two places might be more effective to use each hand to deal at the same time and shoot at two places rather than shooting with one hand each time at one place. Practicing such a game might be very important in patients with neurological motor problems or in patients with a learning disability.

In embodiments of the present invention, one or more flashes are presented to the player. The flash can be randomly presented, activated by the player, activated by the occurrence of predetermined events in the game, or at any other predetermined stage of the game. The flash can be randomly presented, or the player can request such a flash.

In another embodiment of the invention, the game of two opponents can be based on a set of criteria that each player undertakes based on the opponent characteristic. Each player aims to anticipate the opponent responses. Anticipation is approached in this patent as an internal representation that mimics what thinks the enemy's in his/her internal state based on the opponent characteristics, emotional state, and by using knowledge of tactics to select what it would do if it were the opponent. The response can have different facets and the opponent has to beat the player by predicting the next action (for example, chess game). This anticipation can be achieved by encoding many tactics that originally required specialized knowledge. During the game, the characteristics of each player are updated base on the game results and physiological measurements.

In a non-restricting example, during the game, a player has to pick-up armor distributed on screen 120 and shoots the opponent. One player is the attacker and the second player is the defender, wherein the difference between the attacker and the defender is that the attacker has the priority to be the first to select a weapon. During the game, the various armors change disposition and are invisible; the armors generate recognizable sounds so as to indicate to each player what arms are available and the arms location. The player selects a weapon and tries to pick up the selected based on the mental imagery visual percept and the sound percept to be the first to shoot at his/her opponent, a shot that will match the severity of the opponent chosen armor. As weapons vary according to their range, accuracy, the spread of damage, time to load type of ammo and alike, each opponent has to take in consideration in his/her guess the weapon chosen and how should he respond. If either of the players is not hit in the first round, each player can attempt to shoot the opponent in next round, based on the 1D or 2D feedback sound location of the opponent. Following steps are logically chosen based on previous one and on the results obtained.

In embodiments of the present invention, the game rules can be updated by personal physiological variables evaluations, somato-sensory information and perception of each player. The information is preferably available to both players, thereby allowing each player to “learn” the behavior of the opponent.

As the game is played without visual game stimuli, visually impaired player can play the game by mastering the rules, given in the pre-game activity, using auditory signals. For example, the boundary of screen 120 can be learned by having the player move the mouse pointer around till it touches the boundary of screen 120, thereby defining the “ground play” area and developing a mental image of the game space. Such games can be played by one or two “players” wherein the second player is optionally computer 150.

Optionally, the player gives pre-recorded speech remarks to the other player, for example by moving the mouse and pressing on it, by moving the joystick or by touching items on the non-visible screen (yet touch screen) stimuli. Computer 150 may respond with pre-recorded remarks, thus, extending the auditory communication between the player and the game.

More game examples, embodied according to the present invention, will now be described. It should be noted that the present invention is not limited to the described games and any game, performed on a computerized system and including the following steps, is within the scope of the present invention:
a) an act of pre-game activity including learning rules of the game including observing an object on the display thereby forming a visual percept of the object, and transforming the visual percept of the object into a mental image visual percept of the object in the absence of a visual percept of the object; and
b) playing the game, by:
   i. activating the one or more input devices and inputting data in dependence upon the mental imagery percept of the object, in the absence of displaying the one or more objects for a duration of longer than T_{f}, wherein T_{f} is shorter than a time required for an eye movement between two fixation targets and long enough such that the location of said one or more objects can be visually perceived, thereby forming play input data;
   ii. receiving feedback that corresponds to the play input data; and
   iii. activating the one or more input devices and inputting data to form additional play input data in dependence upon the feedback.
      During the game activity, based on memory and mental imagery, each player learns the game rules using input devices such as joystick, keyboard, tactile hand, vibrator motor activities and converting values between psychological, physiological and physical values.

Preferably, each player speaks to a microphone at the pre-game activity or immediately after the beginning of the game and the voice parameters of the player are recorded by the computerized system. The recorded voice parameters then serve as an emotional state baseline of the player.

A game can be played by any number of players and when a game, throughout this innovation specifications, is described in terms of one or two players, it should be noted that the games of the present invention is not limited to be played by one or two players, and can be played by one or more players.

The extent of the inventions is then limited by the skill and imagination of the designers of the game. For example, in war games, the opponent can be another player or computer 150 and attacks can be against targets located in the outer space, in space, on the ground or under water and can be stationary or moving. Likewise, the players can choose different tools such as knifes, arrows or war tools that include classical tools such as swords, pistols or guns, missiles, bombs, but it can also include new war tools such as leasers, electro-magnetic devises or any other device. In a non-restricted example, the war games can incorporate the player or the opponent signals that represent different emotions and act accordingly, so that the player or the opponent can change the personal emotional state to respond in different ways and means.

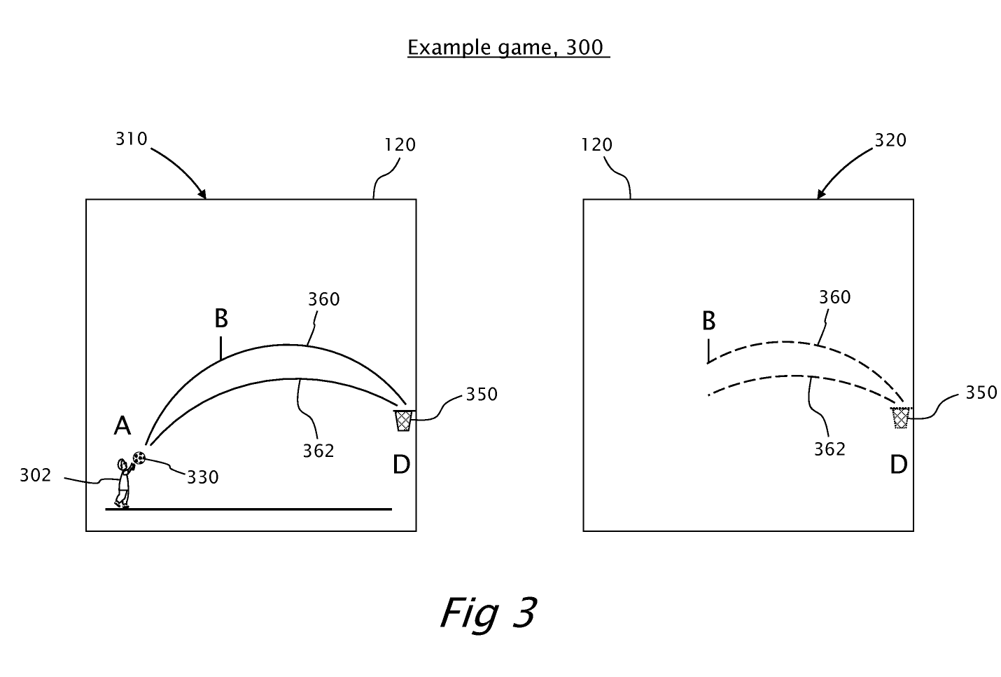

FIG. 3 illustrates an example game 300 based on time prediction of a single event, according to embodiments of the present invention. Pre-game panel 310 shown on screen 120, character 302, representing the player that throws ball 330 from position “A” toward ring 350 placed at point “D”, typically on the other side of screen 120. During game 300, after ball 330 departs from point “A”, and the ball trajectory is clear to the player, the image of ball 330 is removed from screen 120 and game 300 starts, as shown in panel 320.

The payer has to guess when ball 330 will enter the ring from a single event. The player has to predict the time when ball 330 will reach the mental imagery visual perceived ring and press on the keyboard at the expected time. Time estimation should take in consideration the trajectory of ball 330, the velocity of ball 330 and the distance of ball 330 to ring 350, thereby predict the time that ball 330 will enter ring 350. The player challenge is to change prediction when the trajectory changes because ball 330 is thrown higher or lower, and based on the trajectory, the distance to ring 350 and its velocity (determined by the trajectory) changes and therefore the predicted time changes accordingly.

Once the player masters the non-linear interrelationship between the distance, time, velocity and height of ball 330, game 300 begins. The throwing of ball 330 is marked by a feedback tone and the arrival of ball 330 inside ring 350 is marked by another feedback tone. The player has to press a designated key or button when the player thinks that ball 330 enters ring 350. Panel 320 illustrates game 300 at the starting moment. At unpredictable time, when ball 330 reached point “B”, ball 330 vanishes from screen 120. In consecutive trials ball 330 can be thrown at different heights, for example, as shown in panel 320, interrupted lines 360 and 362 represent possible paths for ball 330 to reach ring 350. The player time prediction has to be adapted to the expected trajectory.

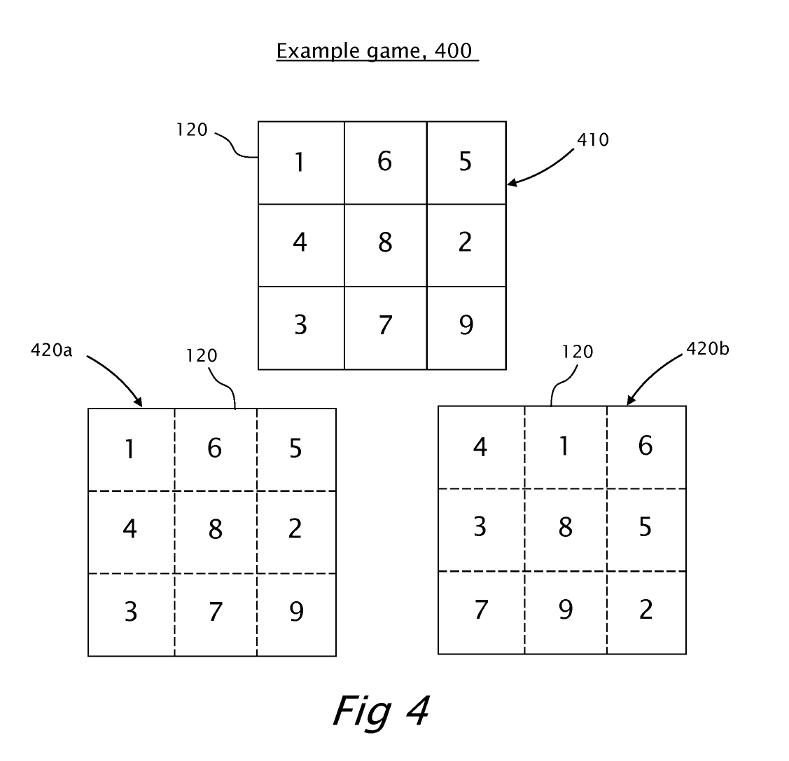

FIG. 4 illustrates a panel 410 which shows an example 3×3 magic square which is revealed and memorized during the pre-game activity and panels 420 reconstructed during game 400, according to embodiments of the present invention. Panel 410 shows the pre-game activity screen when the visual stimuli are presented. Panel 420a shows the reconstructed 3×3 magic square during game 400 (interrupted lines), when the content of the 3×3 magic square is removed from screen 120. In a non-restricted example, in the pre-game activity, the screen is divided in equally visible cells. Computer 150 is programmed to determine and display 9 graphical items, such as numbers, letters, names, symbols, icons and other graphical items, in a pre-determined sequence, wherein one or more items are disposed in each cell of the 3×3 magic square. In variations of the present invention, to reinforce the mental image visual percept, each time that a new item is presented, a tone is delivered so that the player is aware of the presentation, the sequence and its content.

Preferably, in game 400, the player reconstructs the 3×3 magic square, visually presented in the pre-game activity, the player has to “rotate” the squares (around the center), counter clockwise or clockwise, as shown (interrupted lines) in panel 420b. After the rotation, each time that an auditory tone is given, the player has to move the mouse pointer to the correct (rotated) cell and sequentially, press on the keyboard the correct number. Alternatively, the player has to write the symbol or the item that the number represents, as shown in the pre-game presentation. The player tension can be increased if the time between the tones is shortened.

In embodiments of the present invention, during the pre-game activity, the numbers in each cell are shown to the player one at a time, in a pre-determined order and a specific sequence. Simultaneously with the numbers, through the loudspeaker the numbers are correlated with words, symbols or items as heard on the loudspeaker. During game 400, the player has to match the mental perceived image number location with the word, symbol or item associated with each number, and respond by bringing the mouse pointer to the appropriate location of the correlated number on the screen, or if a touch screen is used to touch the screen on the location of the mental imagery visual perceived number and type on the keyboard button the related names to the numbers. During game 400, mental imagery visual percept has to complement the lack of visual percept.

Game 400 can be played by two or more players. When the game starts and the tone is heard, each player takes turns to fill one cell every time a tone is heard. The filling of the cells has to be in the same pattern and order that the cell fillings were shown during the pre-game activity. If one of the players does not respond during the pre-determined time period or makes a mistake, the player misses a turn to the other player. The two players' game can be played either when the instructions are of repeating the symbols in the cells, or when the game rules require that shifting of the numbers of the outer numbers of the magic square should take place. Additional stress on the players can be applied if the time between the tones is delivered at shorter intervals. In the post-game, the correct location of the pre-recorded symbols can be presented together with the player answers and their individual scores.

It should be noted that in game 400, the 3×3 magic square is given by way only and any size and shape of magic squares can be used.

In variations of the present invention, game 400 includes one or more magic squares.

In variations of the present invention, game 400 includes one or more magic squares and when game 400 starts, the one or more magic squares are presented to the player at least partially vacant and in a different orientation and/or scale with respect to the corresponding magic squares presented at the pre-game phase. For example, if the magic square was presented in the pre-game in an upright position, the magic square can presented in the game in a 45° angle orientation, or any other angle.

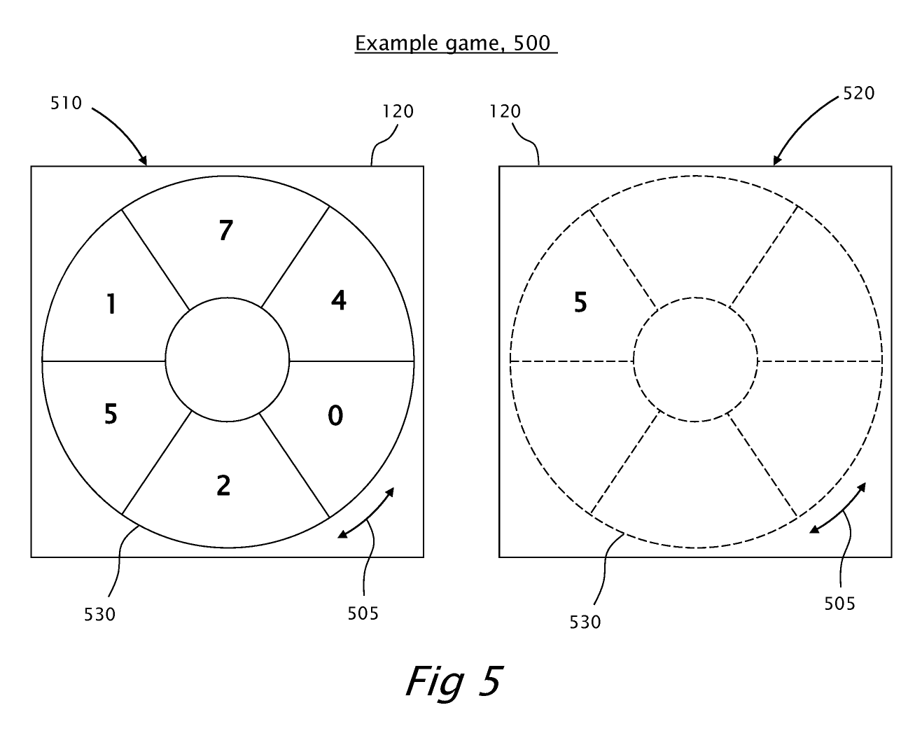

FIG. 5 illustrates a roulette of numbers which are revealed and the order of the numbers are memorized during the pre-game activity and then reconstructed during game 500 using mental imagery visual percept, according to embodiments of the present invention. A roulette 530 is shown with several numbers or other symbols as shown in panel 510. After a pre-determined period of time, the wheel starts rotating in either direction 505. When roulette 530 is stopped, only one number is revealed as indicated (interrupted lines) in panel 520. After a pre-determined period of time, the roulette is stopped and the content of roulette 530 is removed from screen 120 and game 500 starts. The player has to reconstruct the numbers of roulette 530 in the appropriate order using mental imagery visual percept, and such that the revealed single number is disposed at the location where the revealed single number was revealed. In embodiments of the present invention, when roulette 530 rotates, the content of roulette 530 is removed from screen 120 and a tone followed by a word indicates the location and the number. The player has no visual percept of the game where to start and he has to base the answer on the rotated mental imagery visual percept.

In another embodiment of the roulette game, during the game, when the roulette is shown but no numbers are reveled, the player has to “guess” the location of one number and based on the guessed location, to complete the roulette numbers using memory and mental image percept.

It should be noted that there are many variations to game 500. For example, the numbers can be replaced by associated visually perceived items or to relate the numbers to mental image auditory perceived items. During the game, the player does not have visual perceptual reference of the location, only a two dimensional tone that indicates the position.

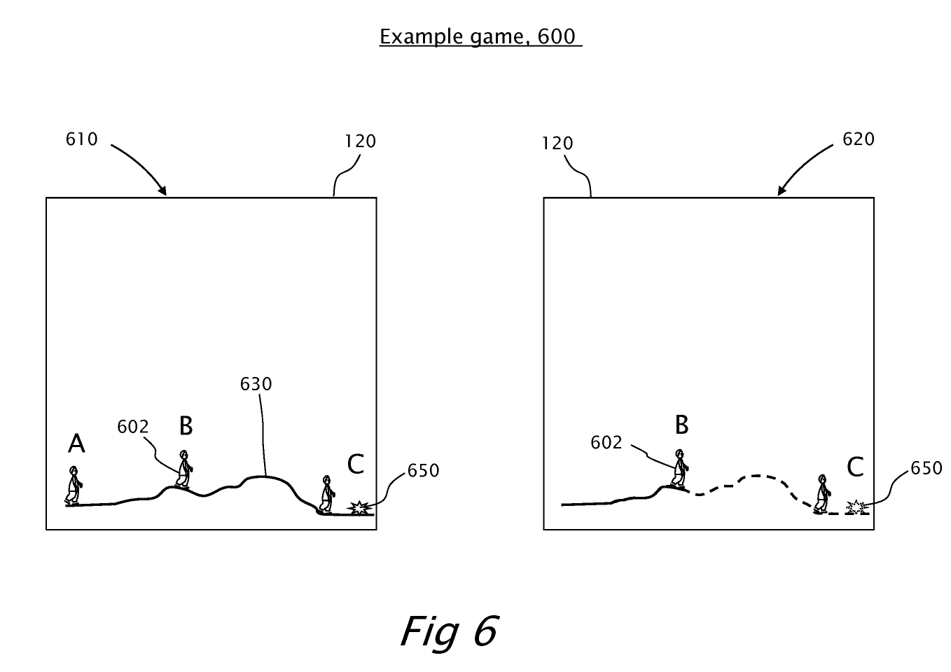

FIG. 6 shows game 600 character 602 walking on terrain 630 to a target 650, which is practiced during the pre-game activity and then played from visual perceived mental imagery, according to embodiments of the present invention. Game 600 is based on converting speed and distance to time. In panel 610, character 602 walks from point “A”, typically on one side of screen 120, to point “C”, typically at the other end of screen 120, to pick-up an item 650. It should be noted that in a non-restricting example, the speed of reaching target 650 depends on terrain 630 structure and conditions, the type of moving as character 602 can run, crawl, jump, swim, and be confronted with obstacles. In the pre-game activity, the player has to learn the pattern of terrain 630 and the distance between points “A” and “C”. When game 600 starts as shown, in panel 620, character 602 moves and reached an unpredictable point “B” where at least a portion of terrain 630 is removed from screen 120 (interrupted line). Based on memory and the mental imagery visual percept in estimating the speed of character 602, terrain 630 conditions, obstacles that character 602 has to overcome, and the distance to target 650 from the time terrain 630 was removed from screen 120, the player has to predict the time when character 602 will reach target 650.

In embodiments of the present invention, in game 600, character 602 is forced to go in circles, cover several paths, or configure character 602 to move in different segment with different velocities such as walking and running. Hence, the player has to estimate the time for character 602 to reach target 650 based on more variables. Typically, at the end of game 600, the player is shown the correct answers and optionally the correct answers compared with the player's answers.

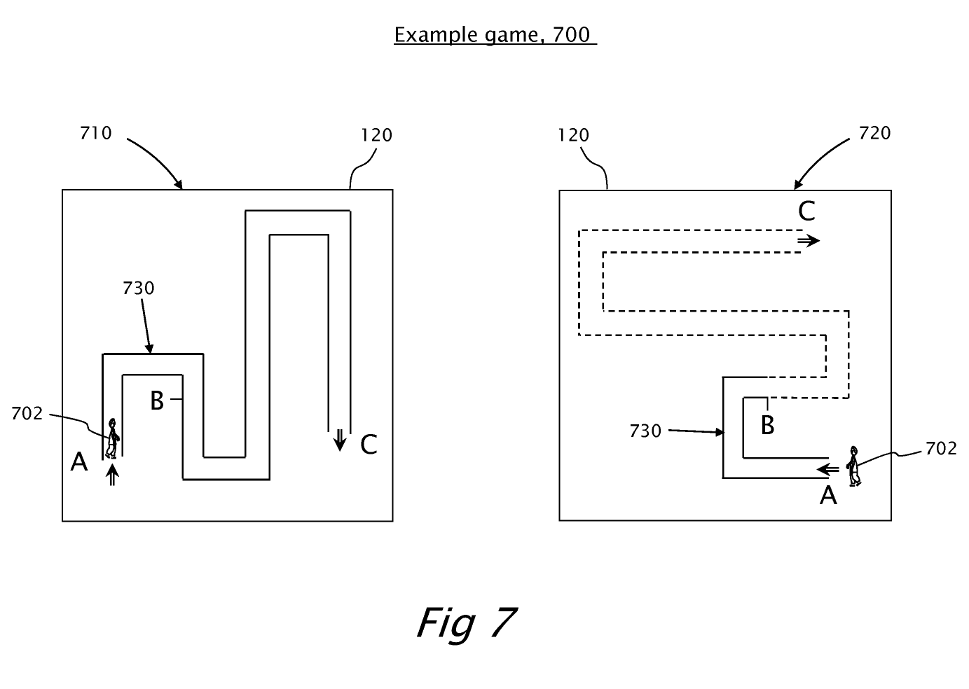

FIG. 7 illustrates maze game 700, which is practiced during the pre-game activity and then played from mental imagery percept and memory, according to embodiments of the present invention. Game 700 is based on converting speed and distance to time. In panel 710, a maze 730 is presented in which character 702 has to walk through from starting point “A” to end point “C”. When character 702 reaches point “C” a tone is sound. During game 700, the moving of character 702 in maze 730 is performed, for example, by moving the mouse pointer along the path of maze 730. If the mouse pointer touches the side of maze 730, a tone is preferably sound. During game 700, the player starts moving the pointer/courser in maze 730 but when character 702 reaches an unpredictable point “B” at least a portion of maze 730 is removed from screen 120 (interrupted lines). In a non-restricting example the speed of reaching the target depends on the terrain condition, the mean of moving as he can run, crawl, jump, swim, and confront with obstacles he has to overcome. In the pre-game activity, the player has to learn the pattern of maze 730, the speed of movement and the distance between points “A” and “C”. When the player reaches point “B”, maze 730 is removed from screen 120 and the game 700 starts as shown in panel 720 (interrupted lines). As shown in panel 720, maze 730 is rotated and the player is informed that maze 730 has been rotated. Optionally, the player may or may not be informed as to the angle of rotation and the direction, and thus, the player has to guess the correct path. Character 702 moves forward in order to reach the end of maze 730 at point “C”. The movement speed of character 702 is based on the mental imagery visual percept of maze 730. The player has to predict the time when character 702 will reach the end of maze 730 at point “C”. It should be noted that the shape, length and obstacle dispersion along the path of maze 730 may change with every trial.

In embodiments of the present invention, in game 700, computer 150 records the duration that it takes character 702 to reach point “C” and report to the player how he performed, for example, he “doubled the speed” compared to the previous trail.

In embodiments of game 700, parameters of the games may change, after maze 730 has been rotated. For example, the speed at which character 702 moves changes, for example: the speed is doubled and the player is informed. Based on the velocity of the image percept, the player has to modify the movement of character 702 along the path of maze 730 to reach point “C” the right time.

In yet another embodiment of game 700, a moving target replaces character 702 and the mouse pointer. At the beginning of game 700, as shown in panel 710, the player presses a designated button and the moving target starts moving from point “A”. The target moves along maze 730 at a fix velocity. At an unpredictable point “B” maze 730 is rotated and removed from screen 120. The player has to predict the time that the moving target will reach point “C” and press a designated button so that pressing the button coincides with the moving target reaching point “C”. Optionally, when maze 730 is removed from screen 120, the player is informed that the velocity of the target has changed. Based on the mental imagery velocity percept, the player has to modify the prediction as to when the moving target will reach point “C”.

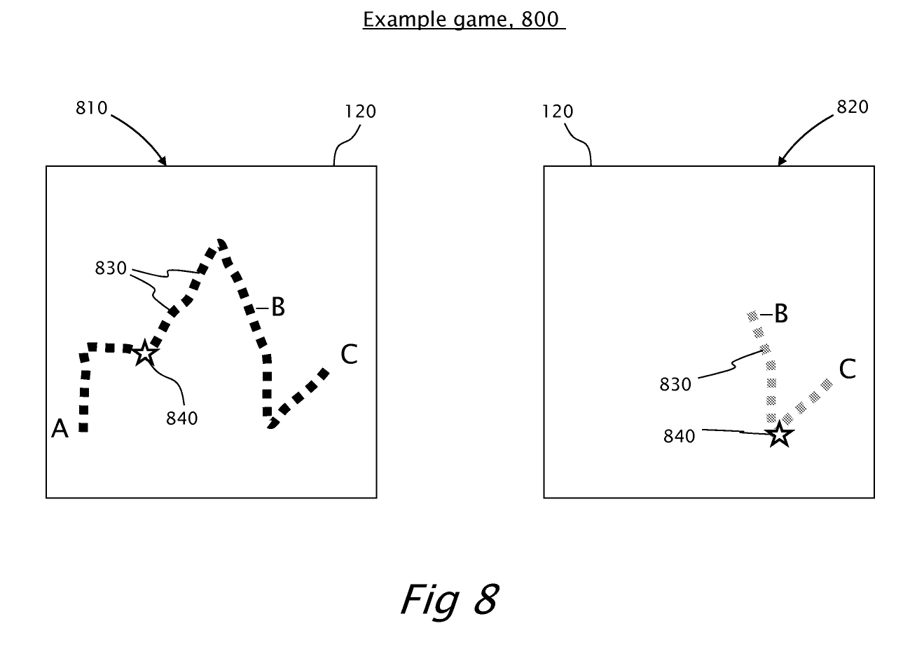

FIG. 8 illustrates a track moving game 800 which is practiced during the pre-game activity and then played based on the mental perceived visual percept according to embodiments of the present invention. In game 800, the player has to estimate time based on equally time spaced discrete repetitive events. For example, in panel 810 moving target 840 moves at fixed velocity along a set of marks 830 equally spaced. Each time target 840 reaches a mark 830, a tone is sound. Game 800 starts at point “A” when the player presses a designated button and ends at point “C”. Target 840 starts moving along the trajectory in a discreet fashion to an unpredictable point “B” where marks 830 are removed from screen 120 and the scale of the marks change, so they can't be reproduce by sheer mental visual percept. Each time target 840 moves to a mark, the player has to press a designated key. The player has to estimate the repetitive time sequence and the distance to the end of the path so that he will press a designated key, when the player estimates that target reaches 840 point “C”. Simultaneous with the player's answer, computer 150 compares the recorded answer with the player answer, and if the player time estimation is longer or shorter by a pre-determined error, a message indicating of the fact is given to the player and the player has to start game 800 from the beginning.

Optionally, after the end of the game, the player is shown on an X-Y plot and/or a table of the correct trials answers compared with 1) the location of the visually perceived point “A”, 2) the location of the visually perceived end point “C”, 3) the location of the marks and when the player pressed on the mouse to mark that moving target 840 passed on them, and 4) the speed and trajectory performed by the player. A score can easily be given based on the evaluation of the variables provided by the plot and/or table.

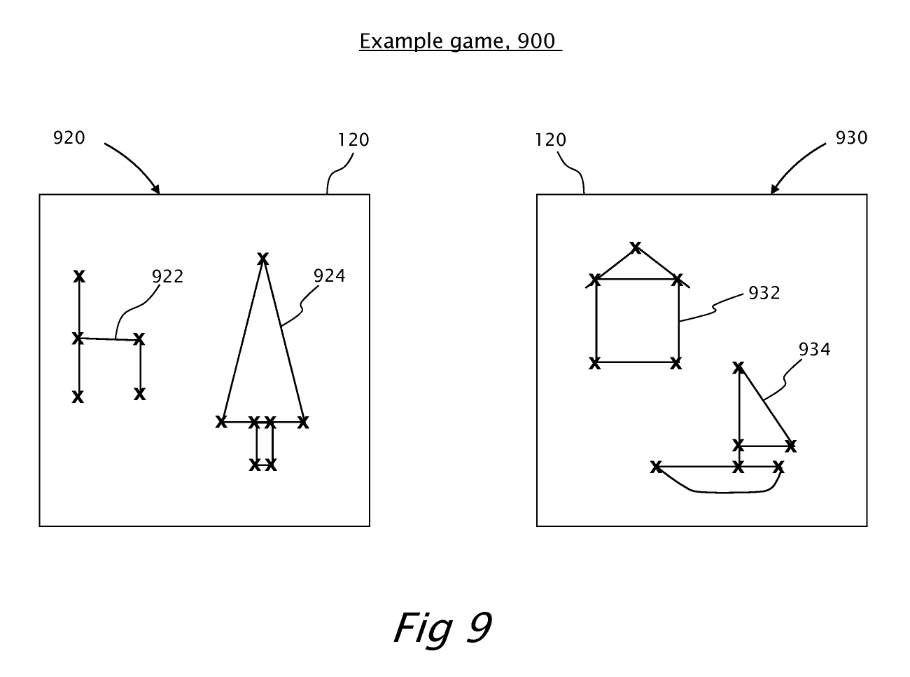

FIG. 9 illustrates redrawing game 900 showing a few example of images which are revealed during the pre-game activity and after changing the scale of the images, the player has to then redrawn from mental image visual percept during the game, according to embodiments of the present invention. To redraw the drawing the player can use the mouse, the joystick or any other device. In panel 920 two examples are shown: Christmas tree 924 and chair 922, and in panel 920 two other examples are shown: house 932 and sailing boat 934. In the pre-game activity, one picture is shown for a pre-determined short duration or a flash. Thereafter, the object to be drawn is removed from screen 120 and the game 900 starts. A dot then appears on screen 120 to indicate where the drawing starts.

To enforce the player to use mental imagery visual percept, the player has to reconstruct the drawing in a different scale or a different scale of just one of the drawings dimensions. The player has to “draw” on the erased screen with a designated device such as the mouse pointer, or the joystick. At points of interest in the drawings, such as intersection of two lines, the touching of two lines, a change in a line direction, etc., (denoted in panels 920 and 930 with “X” marks) the player is informed with a tone, corresponding to the type of point of interest. A curved line is preferably denoted by a special tone. Once the player thinks that he has completed the drawing, the player presses a designated key and the player's drawing, together with the correct drawing, are shown. If the payer greatly misses the drawing, the player's percept can be reinforced by flashing the picture to the player when redrawing.

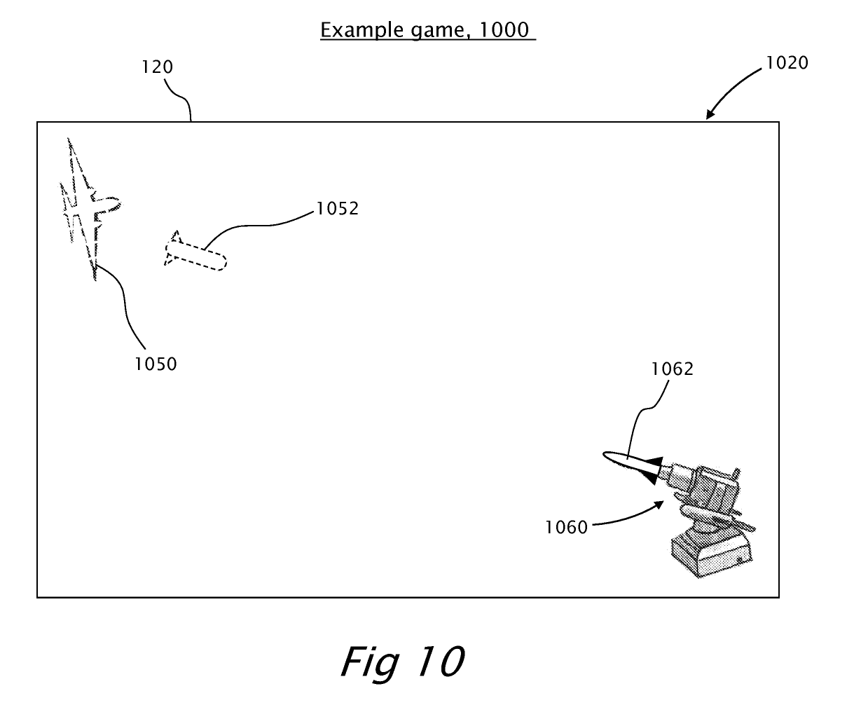

FIG. 10 illustrates game 1000 with missiles 1062 that are trying to hit the moving targets 1050. Game 1000 is practiced during the pre-game activity and then replayed from mental imagery visual percept and memory, according to embodiments of the present invention, whereas the position of moving target 1050 is indicated by auditory cues. In game 1050, the player responses are based on time prediction to either stationary or moving targets. In panel 1020, two players are playing were each player has a manually controlled missile device, wherein one player is the attacker and the second player is the defender, and wherein the difference between the attacker and the defender is that the attacker has the priority to be the first to shoot. The player has to shoot and destroy the opponent. In the non-restricted example shown in panel 1020, one controlled missile device is a stationary missile launcher 1060, and the second controlled missile device is airplane 1050, wherein and missile 1052 by airplane 1050 against missile launcher 1060. During game missile launcher 1000, either the attacker (airplane 1050) or the defender (launcher 1060) or both, attacker and defender, are removed from screen 120, and the locations of the removed items are each indicated by designated tones having different pitch and/or intensity. In panel 1020, airplane 1050 (attacker) and corresponding missile 1052 are marked with interrupted lines to indicate that they vanish from screen 120 during the game.

Typically, at the beginning of the game, both players hear short repetitive tones with different pitches and intensities indicating the location of launcher 1060 and plane 1050. When the attacker shoots, the moving missile generates an interrupted sound and the defender has time to plan to shoot back a missile. During the flight of both missiles (1052, 1062), each player can maneuver and change the flying missile trajectory. Each player can attempt to shoot at the missile or at the targets. If a player aims to shoot the opponent's missile, when in the proximity of the other missile, the player has the option to explode it, hoping that the explosion will destroy the other player's missile. Optionally, game 1000 can be played by one player against computer 150.

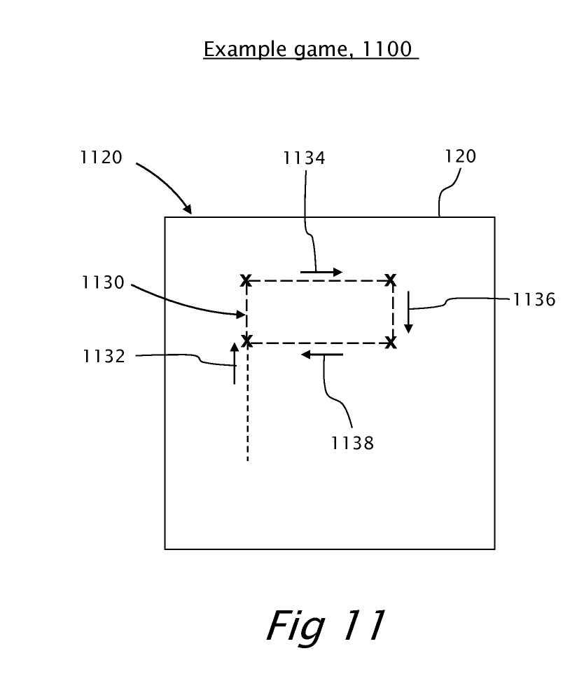

FIG. 11 illustrates game 1100, according to embodiments of the present invention, in which the player has to convert two dimensional auditory signals to a mental imagery visual percept and move his/her hand based on this image. Before game 1100 starts, a dot indicating the starting point is shown. Game 1100 starts with training the player to identify an object and the object's location, using only auditory clues. In a non-restricting example, a continuous sound getting stronger in intensity at high pitch indicates drawing a line in the upward direction (1132), and getting weaker in down direction (1136). If the continuous sound is getting stronger and has a lower pitch, the sound indicates drawing a line from left to right (1134), or getting weaker with the same pitch—drawing a line from right to left (1138). If at some point, two short tones are superimposed on the sound, it indicates that the current line being drawn is crossing a previously drawn line, and if there is only one tone, the current line is touching a previous drawn line.

Hence, in the example shown in FIG. 11 (interrupted lines), the auditory sequence should be as follows:
a) a continuous sound getting stronger in intensity at high pitch, line in up direction (1132) and one short superimposed tone indicating the end of the line;
b) a continuous sound with the same intensity with low pitch, line to the left (1134) and one short superimposed tone indicating the end of the line;
c) a continuous sound getting weaker with high pitch, line in down direction (1136) and one short superimposed tone indicating the end of the line; and
d) a continuous sound with the same intensity line in left direction (1138) at high pitch and two short superimposed tones indicating the touch to another line and the end of the line.
   When the sounds stop, the player should enter the name of the item drawn. Thus, based on two dimensional auditory signals and mental image auditory percept, an item can be identified.

In embodiments of the present invention, game 1100 can be further complicated by introducing additional auditory signal that indicate other types and features of lines, such as a curved lines and interrupted lines.

In embodiments of the present invention, auditory sound percepts have to be converted to mental imagery vision percept and to written words. The auditory percept is used to play a quiz where sound can be converted to letters and words, i.e., to identify words specified by auditory signals. The auditory signal is specifies by seven characteristics learned in the pre-game activity: a continuous sound is heard shifting gradually from left to right and vice versa in a slow repetitive movement. The sound is randomly interrupted about every 8-10 sec. In some of the interruption gapes a short tone is given, 0.3-0.5 sec in the middle of the gap. The continuous auditory characteristic are: direction of the sound (left or right), the presence of the interruption gap, the presence of a tone in the gap with different intensities (high or low) and tone frequency (high or low pitch). Those parameters represent 7 conditions that can be coded into seven letters, each representing one sound condition. Using such a code, letters can be formulated so the player has to identify the word that he has to type it on the keyboard. The player is challenged to find the word. The game can be further expended by having the words form a phrase or a saying.

During the game, a sound and tones are delivered and according to the learned codes during the pre-game activity, the player has to convert the auditory signal to letters. There are numerous ways to complicate the game for advanced player. For example, to teach a set of related keys that are less than the number of auditory variables; the player has to “guess” one of the several keys to answer correctly. For wrong answer (or wrong “guess”) the player can correct but he might lose points in the score. In a non-restricting example, the game can be played between two (or more) players that “communicate” between them were the letters that the player types on the keyboard are converted by computer 150 to auditory codes. Thus, such a game is ideal to be played on the phone, Internet or any technical means that auditory signal can be passed between the players, particularly when they try to “code” their discussion.

Optionally, in the post-game period, the player is presented with the game results, for example, by a colored two dimensional graph can be displayed that includes the correct answer with the player's answers. The comparison between the graphs can provide the players with verification of their typed dialog and the appropriate score.

In another embodiment of the game, auditory sound percepts have to be converted to mental imagery vision percept and to written words. The player has to recall words and their spelling. The aim in this game is to spell the recalled auditory signals to increase player vocabulary, and decrease the spelling mistakes. The game can be played by healthy player or patients who suffer from a learning disability. In the pre-game activity, the player is shown a puzzle (for example, a 3×3 size) with few letters as “filling spaces” or clues, and numbers as is usually presented in a puzzle. He hears a word, its number in the puzzle and the direction (horizontal or vertical). During the game, he has to remember the location of the numbers and the additional letters presented. Following the heard word, he has to answer by typing the word with the keyboard. When he finishes writing, he should press on the keyboard and then the next word is given to him by an auditory signal.

The game can be easily complicated by giving the player a short description of a profession or an act as the words he has to look for to be placed in the puzzle. For example, he can be asked to write from a specific number and direction the profession of the person who is “sewing cloths”, or who is “taking care of a garden”.

In another modification of the game, the player can construct during the game a puzzle, either with the help of an auditory signal or without.

After the game, it's easy to present the puzzle with the pre-game letters and the correct answers with color and with the player answer and evaluate the score in the game.

Games, rules and classification of the conditions given during the pre-game activity and the game, as well as the speech analysis, emotional state criteria and the correct answers for post-game are pre-recorded in any storage media.

The games should be considered only as non-restricted examples as they can easily be changed to apply to a wide range of games and to cover such fields as combat games (skill and action game), maze games, sports games, ping-pong games, race games, strategy games (emphasize cognition rather than manipulation), deadline games (detective adventures with or without obstacles), war games, game of chance, educational and children's games.

Although the present invention has been described with reference to the preferred embodiment and examples thereof, it will be understood that the invention is not limited to the details thereof. Various substitutions and modifications have suggested in the foregoing description, and other will occur to those of ordinary skill in the art. Therefore, all such substitutions and modifications are intended to be embraced within the scope of the invention as defined in the following claims.

What is claimed is:

1. In a computerized system, including a display, storage capacity and one or more input devices, a method for playing a computer game by one or more players, the method comprising:
a) an act of pre-game activity including learning rules of the game including observing one or more objects related to the game on said display, thereby forming a visual percept of said one or more objects, and transforming said visual percept of said one or more objects into a mental image percept of said one or more objects in the absence of a visual percept of said one or more objects; and
b) an act of game activity including:
i) activating said one or more input devices and inputting data in dependence upon said mental imagery percept of said one or more objects in the absence of displaying the one or more objects for a duration of longer than T_{f}, wherein T_{f} is shorter than a time required for an eye movement between two fixation targets and long enough such that the location of said one or more objects can be visually perceived, thereby forming play input data;
ii) receiving feedback that corresponds to said play input data; and
iii) activating said one or more input devices and inputting data to form additional play input data in dependence upon said feedback.

2. The method as in claim 1, wherein said one or more input devices are selected from the group of peripheral devices including a keyboard, a joystick, a mouse, a game pad, a push button and commonly known computer games input devices.

3. The method as in claim 1, wherein said one or more act of game activity further includes a flash displayed for a duration T_{f}, wherein T_{f} is shorter than a time required for an eye movement between two fixation targets and long enough such that the location of said one or more objects can be visually perceived.

4. The method as in claim 1, wherein said feedback is provided to said player in a form selected from the group consisting of an audio signal, a textual feedback displayed on said display, activation of a light indicator and activation of a vibrator of said one or more input devices.

5. The method as in claim 1, wherein said act of pre-game activity further includes a step of recording the voice of said player into said memory, thereby creating an emotional state baseline of said player.

6. The method as in claim 5, wherein said voice parameters of said player are selected from the group including: pitch, amplitude, pace, rhythm and prosodic.

7. The method as in claim 5, wherein said game activity includes the steps of:
a) recording the voice of said player into said memory, thereby obtaining an emotional state of said player;
b) comparing said emotional state of said player with said emotional state baseline of said player, to determine a change in emotional state of said player; and
c) modifying said game rules using said change in emotional state of said player.

8. The method as in claim 1 further comprising the step of real-time monitoring of physiological parameters related to said one or more players' physiology, wherein said game rules are modified in dependence upon said physiological parameters.

9. The method of claim 1, wherein one of said players is the host computer.

10. The method of claim 1, wherein said act of game activity, further includes the step of selecting and operating at least one of said input devices by said player, in particular in games that demand performance of dual-task.

11. The method of claim 1, wherein said act of game activity, further includes the step of measuring at least one physiological monitoring tool selected from the group including: respiratory rhythms, heart rate, blood pressure, tactile, resistance skin conductance, encephalogram and electro-myograph.

12. The method as in claim 1, wherein said computer game is a target hitting game, wherein said object is a stationary or moving target and wherein said act of game activity further includes the steps of:
iv) activating speakers to sound auditory signals, said auditory signals associated with the spatial location of said target; and
v) operating at least one of said input devices by said player to activate a weapon to launch ammunition to hit said target.

13. The method as in claim 12, wherein said target is selected from the group consisting: ground targets, air targets, outer-space targets and underwater targets.

14. The method of claim 1, wherein said computer game is a puzzle game having a character associated with said player; wherein said puzzle is rotated and optionally changes position on said screen and optional scaled, before said act of game activity begin; and wherein said act of game activity further includes the steps of:
iv) providing said player with different pre-stored questions and phrases such as syllables, words, items, pictures, auditory grapheme or phoneme and sounds to increase the skills in learning in general and learning languages in particular; and
v) providing an answer by said player, wherein the correct answer is provided to said player by an auditory signal immediately after said answer of said player or at the end of said game.

15. The method of claim 1, wherein said computer game is a drawing game having a drawing pointer associated with said player, wherein said act of game activity further includes the step of operating at least one of said input devices by said player to activate said drawing pointer to draw a drawing task without visual percept of at least a portion of said drawing.

16. The method of claim 15, wherein said drawing task includes changes in the drawing, selected from the group including dimensions and orientations.

17. The method of claim 1, wherein said computer game is a racing game race between two or more of said players, and wherein said one or more objects include a racing object associated with each of said players, wherein said act of game activity further includes the step of operating at least one of said input devices by each of said players to activate said racing objects.

18. The method of claim 17, wherein said act of game activity further includes the step of activating speakers to sound auditory signals, wherein said auditory signals are respectively associated with the spatial location of said racing objects, and wherein said auditory signals are facilitated to change in intensity, pitch, or frequency.

19. The method of claim 17, wherein said act of game activity further includes the step of activating a vibrator to provide tactile information, said tactile information associated with the spatial location of said racing objects.

20. The method of claim 1, wherein said computer game is a ball game having a character associated with said player, wherein said act of game activity further includes the step of operating at least one of said input devices, by said player, to activate said character to bounces said ball with a controllable force to reach a target location, and wherein the surface on which said ball is bouncing has different and changeable elasticity properties.

21. The method of claim 1, wherein said computer game is a maze having obstacles and a character associated with said player, wherein said maze is rotated and optionally changes position on said screen and optional scaled, before said act of game activity begin, and wherein said act of game activity, further includes the step of operating at least one of said input devices by said player, and wherein said obstacles force said character to walk, bent, run, jump, crawl, swim or shoot in a certain order and certain direction.

22. The method of claim 1, wherein said act of game activity further includes the step of providing each of said players with a flash of visual percept of said display, and wherein said flash is activated by the player, randomly by said game or at the occurrence of a predetermined event in said game.

23. The method of claim 1, wherein said computer game is a combat game having a character associated with said player, wherein said act of game activity further includes the steps of:
iv) activating speakers to sound auditory signals, said auditory signals associated with the spatial location of each of said characters;
v) operating at least one of said input devices by said player to select a weapon, to be carried by a respective character of said player; and
vi) operating at least one of said input devices by said player to activate said character to operate said weapon and move, including moving, jumping, crawling, swimming, air parachuting or hiding; and wherein said combat situated in a space domain selected from the group consisting: on the ground, at the air and underwater.

24. In a computerized system, the system including a display, memory and one or more input devices, a method for playing a computer game by one or more players, the method comprising the steps of:
a) an act of pre-game activity including learning rules of the game including observing a moving object on said display and in the absence of a visual percept of said moving image, transforming said visual percept of image of said moving object into a mental image percept of said moving object; and
b) playing the game, by
i) using said one or more input devices and inputting data in dependence upon said mental imagery percept related to game play in the absence of the observing the moving object for a duration longer than T_{f}, wherein T_{f} is shorter than a time required for an eye movement between two fixation targets and long enough such that the location of the object can be perceived;
ii) receiving feedback that corresponds to said input data; and,
iii) using said one or more input devices to input additional data in dependence upon said feedback.

25. The method of claim 24 further comprising the step of providing flash information displayed for a duration T_{f}, wherein T_{f} is shorter than a time required for an eye movement between two fixation targets and long enough such that the location of said moving object can be perceived.

26. In a computerized system, the system includes a display, memory and one or more input devices, a method for playing a computer game by one or more players, the method comprising the steps of:
a) providing on said display a moving object;
b) allowing sufficient time for said one or more players to form a visual percept of said moving object;
c) removing said moving object from said display thereby forcing said one or more players to transform said visual percept of said moving object into a mental image percept of said moving objects;
d) in the absence of the moving object prompting the said one or more players for input data related to said playing of said computer game;
e) providing said one or more players feedback that corresponds to said input data; and
f) prompting the said one or more players for additional input data in dependence upon said feedback.

27. The method of claim 26 further comprising the step of providing flash information displayed for a duration T_{f}, wherein T_{f} is shorter than a time required for an eye movement between two fixation targets and long enough such that the location of said moving object can be perceived.
